(19) United States
(12) Patent Application Publication
(10) Pub. No.: US 2019/0086392 A1
(43) Pub. Date: Mar. 21, 2019

(54) METHODS FOR DIAGNOSIS AND TREATMENT OF SOLAR LENTIGO

(71) Applicants: INSERM (INSTITUT NATIONAL DE LA SANTE ET DE LA RECHERCH MEDICALE), Paris (FR); UNIVERSITE PARIS XIII PARIS-NORD, Villetaneuse (FR); UNIVERSITE DE FRANCHE-COMTE, Besancon (FR); DERMANCE, Paris (FR); CHRU DE BESANCON, Besancon (FR); EFS BOURGOGNE FRANCHE COMTE, Besancon (FR)

(72) Inventors: Christine LE ROY, Bobigny (FR); Ranesha GOOROCHURN, Besancon (FR); Nadine VARIN-BLANK, Bobigny (FR); Philippe HUMBERT, Besancon (FR); Céline VIENNET-STEINER, Besancon (FR)

(21) Appl. No.: 16/086,337

(22) PCT Filed: Mar. 20, 2017
(86) PCT No.: PCT/EP2017/056591

§ 371 Date: Sep. 19, 2018

(30) Foreign Application Priority Data
Mar. 21, 2016 (EP) 16305314.3

(51) Int. Cl. G01N 33/50; G01N 33/68

(52) U.S. Cl. CPC G01N 33/5041; G01N 2333/5437; G01N 2800/207; G01N 33/6869

(57) ABSTRACT

The present invention relates to the diagnosis and treatment of solar lentigo (SL). The inventors established an in vitro model of primary fibroblasts isolated from SL (FL) and perilesional (FS) biopsies, which were collected from a cohort of 10 volunteers. Then, the inventors defined morphological and functional characteristics of both dermal cells. The inventors demonstrated by immunofluorescence studies differential morphological features with FL displaying elongated shape, a thin epidermis, disorganized basement membrane, intense melanin deposition and elongated rete ridges collapsing into the dermis and FS presented flattened morphology. Moreover, both fibroblasts demonstrated distinct functional characteristics with FL exhibiting a lower proliferation rate and migration capacity, senescent-like phenotype as well as a higher ability to secrete KGF, HGF, SCF, IL-13 and TGFβ1. Thus, the present invention relates to a method of identifying a subject having or at risk of having or developing solar lentigo, comprising measuring the expression level of IL-13, TGFβ1, HGF, KGF and SCF. The present invention also relates to an IL-13 inhibitor compound for use in the treatment of solar lentigo.

### FIELD OF THE INVENTION

The present invention relates to the diagnosis of solar lentigo. The present invention also relates to methods and compositions for the treatment of solar lentigo.

### BACKGROUND OF THE INVENTION

In physiological conditions, human skin undertakes physical protection against external environment factors, such as microorganisms and ultraviolet (UV) radiations, as well as intrinsic components, including inflammation and cancers (1). This protective function is mediated through the epidermis that is mainly composed in its basal layer of melanocytes that synthetize the melanin pigment (2) and adjacent keratinocytes that collect and dispense the pigment to the upper layers of the skin (3-5). The underlying dermis includes fibroblasts, which mostly produce extracellular matrix components, and immune cells (6, 7). While the molecular and cellular events controlling pigmentation are not completely elucidated, increasing evidence point out the role of fibroblasts in impacting the functions of melanocytes and keratinocytes by secreting various factors in response to numerous signals from the epidermis (1, 8, 9). The influence of dermal fibroblasts is highlighted through in vitro reconstructed pigmented skin models, in which normal fibroblasts play a crucial role in the regulation of the melanogenesis process (10-12). Interestingly, converted fibroblasts are at the origin of functional melanocytes (13).

Dysregulation of skin homeostasis manifests as altered pigmentation processes, such as hyperpigmentation in the benign Solar Lentigo (SL). This lesion increases with age and results from chronic exposure to UV radiations and pollution (8, 14-20). Defined histologically by a higher melanin deposition in the basal layer, elongated epidermal ridges and large melanosomal complexes (12, 21, 22), SL is distinguishable from other hyper-pigmented diseases (14) and classified in function of its evolution (23, 24). Differential gene-profiling analyses between SL and normal skin biopsies revealed that SL tissues are mainly composed of activated melanocytes as well as hypo-proliferating and -differentiated keratinocytes on the background of chronic inflammation (8, 25, 26). Transcriptomic and proteomic studies begin deciphering the network of soluble factors at the tissue scale and cellular levels involved in the initiation and formation of SL spots (8, 27). Intense staining for HGF (Hepatocyte Growth Factor), KGF (Keratinocyte Growth Factor), SCF (Stem Cell Factor) and sFRP2 (secreted Frizzled Related Protein 2) in the upper dermis of SL biopsies strongly suggests that dermal fibroblasts contribute to synthetize these factors (28, 29). These data were supported by their transcriptional up-regulation in an in vitro model of photo-aging induced fibroblasts, evoking their potential functional paracrine involvement (28). Analysis of a 3D reconstructed skin model containing epidermal and dermal cells showed higher levels of pigmentation in tissues with natural photo-aged fibroblasts vs young photo-protected fibroblasts, suggesting that both types of fibroblasts communicate with their neighbouring epidermal cells through differential secreted protein networks (10). Surprisingly, among the quantified soluble factors known to modulate pigmentation, such as HGF and KGF, solely GM-CSF (Granulocyte Macrophage-Colony Stimulating Factor) secretion level was significantly decreased in photo-aged fibroblast conditioned media (10), suggesting functional differences between in vitro photo-aged fibroblasts and SL fibroblasts.

Chronic sun exposure induces skin heterogeneity with the presence of non-lesional and lesional areas. Among these skin alterations, Solar Lentigo maculae (SL) are benign and hyper-pigmented lesions. Despite the recent literature describing some molecular and cellular events involved in the SL development, little is known about the contribution of the dermal fibroblasts.

The inventors established an in vitro model of primary fibroblasts extracted from SL and peri-lesional biopsies, which were collected from a cohort of volunteers. Cultured fibroblasts allowed us to define their differential morphological features and functional characteristics, notably their secretion capacity of cytokines and growth factors that might be biomarkers of the SL.

### SUMMARY OF THE INVENTION

The present invention relates to the diagnosis of solar lentigo. The present invention also relates to methods and compositions for the treatment of solar lentigo.

### DETAILED DESCRIPTION OF THE INVENTION

The inventors established an in vitro model of primary fibroblasts isolated from SL (FL) and peri-lesional (FS) biopsies, which were collected from a cohort of 10 volunteers. Then, the inventors defined morphological and functional characteristics of both dermal cells.

The inventors demonstrated by immunofluorescence studies differential morphological features with FL displaying elongated shape and FS presented flattened morphology; no difference was observed in their myofibroblastic phenotype. Moreover, both fibroblasts demonstrated distinct functional characteristics with FL exhibiting a lower proliferation rate and migration capacity, as well as a higher ability to secrete KGF, HGF, SCF, IL-13 and TGFβ1. For the first time, establishment of primary FS and FL from the same volunteer identified cellular morphological and functional features in SL.

Accordingly, the present invention relates to an IL-13 inhibitor compound for use in the treatment of solar lentigo (SL) in a subject in need thereof.

As used herein, the term “subject” denotes a mammal. Typically, a subject according to the invention refers to any subject (preferably human) afflicted with or susceptible to be afflicted with hyperpigmentation disorders. In a particular embodiment, the term “subject” refers to a subject afflicted with or susceptible to be afflicted with solar lentigo (SL).

The term “solar lentigo” has its general meaning in the art and refers to actinic lentigo, senile, liver spots, old age spots, or “senile freckles”, the most frequent of hyperpigmentation lesions. The term “solar lentigo” also refers to dysregulation of skin homeostasis manifests as altered pigmentation processes, this lesion increases with age and results from chronic exposure to UV radiations and pollution (8, 14-20). Solar lentigo is defined histologically by a higher melanin deposition in the basal layer, elongated epidermal ridges and large melanosomal complexes (12, 21, 22).

The method of the invention may be performed for any type of hyperpigmentation disorder. The term “hyperpigmentation disorder” refers to hyperpigmentary disorders, which are characterized by an abnormal accumulation (apart from tanning) of melanin, which may be cited include solar lentigo (actinic lentigo), melasma, acne-related pigmentation, post-inflammatory pigmentation, lime disease, pigmentation linked to poison ivy or benign facial dyschromia.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “IL-13” has its general meaning in the art and refers to Interleukin 13. The term “IL-13” also refers to cytokine that acts through the heterodimer complex of IL-13 receptor alpha 1 and IL-4 receptor alpha (IL-13Rα1/IL-4Rα complex) to induce activation responses. IL-13 action may also be mediated through IL-13 receptor alpha 2 (IL-13Rα2). Binding of IL-13 to the IL-13Rα1/IL-4Rα complex leads to the activation of several signal transduction pathways, including JAK/STAT signaling pathway (Corren, 2013; Agrawal and Townley, 2014; Kasaian and Miller, 2008; Suzuki et al., 2015).

The term “IL-13 signaling pathway inhibitor” or “IL-13 inhibitor” has its general meaning in the art and refers to a compound that selectively blocks or inactivates the IL-13. The term “IL-13 signaling pathway inhibitor” also refers to a compound that selectively blocks the binding of IL-13 to its receptors (IL-13Rα1/IL-4Rα complex and IL-13Rα2). The term “IL-13 signaling pathway inhibitor” also refers to a compound that selectively blocks or inactivates IL-13, for example by inhibiting the IL-13 and IL-13Rα1/IL-4Rα complex downstream effectors such as tyrosine kinase proteins Janus kinase 1 and 2 (JAK1 and JAK2), protein signal transducer and activator of transcription family (STAT6, STAT3 and STAT1), tyrosine kinase 2 (TYK2), and transforming growth factor β (TGFβ). As used herein, the term “selectively blocks or inactivates” refers to a compound that preferentially binds to and blocks or inactivates IL-13 with a greater affinity and potency, respectively, than its interaction with the other sub-types of the interleukin family. Compounds that block or inactivate IL-13, but that may also block or inactivate other interleukin sub-types, as partial or full inhibitors, are contemplated. The term “IL-13 signaling pathway inhibitor” also relates to a compound that inhibits IL-13, IL-13 receptors and IL-13 downstream effectors expression. Typically, an IL-13 inhibitor compound is a small organic molecule, a polypeptide, an aptamer, an antibody, an intra-antibody, an oligonucleotide or a ribozyme.

Tests and assays for determining whether a compound is an IL-13 inhibitor are well known by the skilled person in the art such as described in Agrawal and Townley, 2014; U.S. Pat. No. 6,214,559.

IL-13 inhibitors are well-known in the art such as illustrated by Agrawal and Townley, 2014; EP1852503.

In one embodiment of the invention, IL-13 inhibitors include but are not limited to Lebrikizumab (MILR1444A); Tralokinumab (CAT-354); Dupilumab (SAR231893/REGN668); Pitrakinra; IMA-638; IMA-026; GSK679586; AMG 317 and compounds described in Agrawal and Townley, 2014; EP1852503; WO0064944; US 20120039872; WO2003/031587; WO2007080174; WO2008/060814; WO2008/060813; WO2008/049098; WO03/080675; WO2005/079755.

In another embodiment, the IL-13 inhibitor of the invention is a compound inhibiting the IL-13 receptors and IL-13 downstream effectors.

Accordingly, the present invention also relates to a compound which is selected from the group consisting of IL-13Rα1 antagonist, IL-13Rα1 expression inhibitor, IL-4Rα antagonist, IL-4Rα expression inhibitor, IL-13Rα2 antagonist, IL-13Rα2 expression inhibitor, JAK1 inhibitor, JAK1 expression inhibitor, JAK2 inhibitor, JAK2 expression inhibitor, STAT6 inhibitor, STAT6 expression inhibitor, TYK2 inhibitor, TYK2 expression inhibitor, TGFβ inhibitor and TGFβ expression inhibitor for use in the treatment of solar lentigo in a subject in need thereof.

The term “IL-13Rα1” has its general meaning in the art and refers to the IL-13 receptor alpha 1.

The term “IL-13Rα1 antagonist” has its general meaning in the art and refers to compounds such as antibodies described in WO2008/060814; WO2008/060813; WO2008/049098; WO03/080675.

The term “IL-4Rα” has its general meaning in the art and refers to the IL-4 receptor alpha.

The term “IL-4Rα antagonist” has its general meaning in the art and refers to compounds such as antibodies described in WO2010/070346; WO2009081201.

The term “IL-13Rα2” has its general meaning in the art and refers to the IL-13 receptor alpha 2.

The term “IL-13Rα2 antagonist” has its general meaning in the art and refers to compounds such as antibodies described in WO2014/072888.

The term “JAK1” has its general meaning in the art and refers to the tyrosine kinase proteins Janus kinase 1.

The term “JAK1 inhibitor” has its general meaning in the art and refers to compounds such as ruxolitinib (INCB018424); GLPG-0634; Anilinophthalazine-based JAK1 inhibitors and compounds described in Norman, 2012; WO2010/135650; WO2011/086053; WO2009/152133; WO2011/068881; WO2011/112662; WO2012/037132.

The term “JAK2” has its general meaning in the art and refers to the tyrosine kinase proteins Janus kinase 2.

The term “JAK2 inhibitor” has its general meaning in the art and refers to compounds such as ruxolitinib (INCB018424), SAR302503 (TG101348), Pacritinib (SB1518), CYT387, AZD-1480, BMS-911543, BMS-91153, NS-018, LY2784544, Lestaurtinib (CEP701), AT-9283, CP-690550, SB1578, R723, INCB16562, INCB20, CMP6, TG101209, SB1317 (TG02), XL-019, Baricitinib (LY3009104, INCB28050); AC-430; CEP-33779 and XL-019 and compounds described in Norman, 2012.

The term “STAT6” has its general meaning in the art and refers to the protein signal transducer and activator of transcription 6.

The term “STAT6 inhibitor” has its general meaning in the art and refers to compounds such as described in WO2003/031587; WO2014182928.

The term “TYK2” has its general meaning in the art and refers to the tyrosine kinase 2.

The term “TYK2 inhibitor” has its general meaning in the art and refers to compounds such as Triazolopyridine Tyk2 inhibitors and compounds described in described in Norman, 2012; DE102009015070; WO2011/113802, WO2012/035039 and WO2012/066061; WO2012/062704.

The term “TGFβ” has its general meaning in the art and refers to the transforming growth factor β.

The term “TGFβ inhibitor” has its general meaning in the art and refers to compounds such as Galunisertib (LY2157299); TEW-7197; LY3022859; IMC-TRI; Fresolimumab (GC-1008); PF-03446962; Trabedersen (AP-12009); Belagenpumatucel-L; Pirfenidone; and compounds described in Herbertz et al., 2015.

In another embodiment, the inhibitor of the invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against IL-13, IL-13 receptors or IL-13 downstream effectors of the invention as above described, the skilled man in the art can easily select those blocking or inactivating IL-13.

In another embodiment, the inhibitor of the invention is an antibody (the term including “antibody portion”).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of IL-13, IL-13 receptors or IL-13 downstream effectors. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in IL-13, IL-13 receptors or IL-13 downstream effectors. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., I. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. No. 5,800,988; U.S. Pat. No. 5,874, 541 and U.S. Pat. No. 6,015,695). The “Hamers patents” more particularly describe production of VHHs in bacterial hosts such as E. coli (see for example U.S. Pat. No. 6,765,087) and in lower eukaryotic hosts such as molds (for example Aspergillus or Trichoderma) or in yeast (for example Saccharomyces, Kluyveromyces, Hansenula or Pichia) (see for example U.S. Pat. No. 6,838,254).

In one embodiment, the antibody of the invention is directed against IL-13, IL-13 receptors IL-13Rα1, IL-4Rα or IL-13Rα2; or IL-13 downstream effectors JAK1, JAK2, STAT6, STAT3, STAT1, TYK2, or TGFβ.

In one embodiment, the IL-13 inhibitor of the invention is an IL-13, IL-13 receptors IL-13Rα1, IL-4Rα or IL-13Rα2; or IL-13 downstream effectors JAK1, JAK2, STAT6, STAT3, STAT1, TYK2, or TGFβ expression inhibitor.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs, which are modified, by processes such as capping, polyadenylation, methylation, and editing, and proteins (e.g., IL-13) modified by, for example, methylation, acetylation, phosphorylation, ubiquitination, SUMOylation, ADP-ribosylation, myristilation, and glycosylation.

An “inhibitor of expression” refers to a natural or synthetic compound that has a biological effect to inhibit the expression of a gene.

IL-13 expression inhibitors for use in the present invention may be based on antisense oligonucleotide constructs. Anti-sense oligonucleotides, including anti-sense RNA molecules and anti-sense DNA molecules, would act to directly block the translation of IL-13, IL-13 receptors or IL-13 downstream effectors mRNA by binding thereto and thus preventing protein translation or increasing mRNA degradation, thus decreasing the level of IL-13, IL-13 receptors or IL-13 downstream effectors proteins, and thus activity, in a cell. For example, antisense oligonucleotides of at least about 15 bases and complementary to unique regions of the mRNA transcript sequence encoding IL-13, IL-13 receptors or IL-13 downstream effectors can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as IL-13 expression inhibitors for use in the present invention. IL-13, IL-13 receptors or IL-13 downstream effectors gene expression can be reduced by contacting the subject or cell with a small double stranded RNA (dsRNA), or a vector or construct causing the production of a small double stranded RNA, such that IL-13, IL-13 receptors or IL-13 downstream effectors expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as IL-13 expression inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of IL-13, IL-13 receptors or IL-13 downstream effectors mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as IL-13 expression inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing IL-13, IL-13 receptors or IL-13 downstream effectors. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Typically the inhibitors according to the invention as described above are administered to the subject in a therapeutically effective amount.

By a “therapeutically effective amount” of the inhibitor of the present invention as above described is meant a sufficient amount of the inhibitor for treating solar lentigo at a reasonable benefit/risk ratio applicable to any medical treatment. It will be understood, however, that the total daily usage of the inhibitors and compositions of the present invention will be decided by the attending physician within the scope of sound medical judgment. The specific therapeutically effective dose level for any particular subject will depend upon a variety of factors including the disorder being treated and the severity of the disorder; activity of the specific inhibitor employed; the specific composition employed, the age, body weight, general health, sex and diet of the subject; the time of administration, route of administration, and rate of excretion of the specific inhibitor employed; the duration of the treatment; drugs used in combination or coincidental with the specific inhibitor employed; and like factors well known in the medical arts. For example, it is well known within the skill of the art to start doses of the inhibitor at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the inhibitor of the present invention for the symptomatic adjustment of the dosage to the subject to be treated. A medicament typically contains from about 0.01 mg to about 500 mg of the inhibitor of the present invention, preferably from 1 mg to about 100 mg of the inhibitor of the present invention. An effective amount of the drug is ordinarily supplied at a dosage level from 0.0002 mg/kg to about 20 mg/kg of body weight per day, especially from about 0.001 mg/kg to 7 mg/kg of body weight per day.

In a particular embodiment, the inhibitor according to the invention may be used in a concentration between 0.01 μM and 20 μM, particularly, the inhibitor of the invention may be used in a concentration of 0.01, 0.05, 0.1, 0.2, 0.3, 0.4, 0.5, 1.0, 2.5, 3.0, 3.5, 4.0, 4.5, 5.0, 10.0, 15.0, 20.0 μM.

In a further aspect, the present invention relates to the inhibitor according to the invention in combination with one or more anti-solar lentigo compound for use in the treatment of solar lentigo in a subject in need thereof.

The term “anti-solar lentigo compound” has its general meaning in the art and refers to compounds used in the treatment of solar lentigo such as depigmenting treatments; compounds reducing melanin synthesis activity in the melanocytes; hydroquinone and hydroquinone derivatives; kojic acid; arbutin; iminophenols; ascorbic acid and ascorbic acid derivatives; carnitine and quinone; aminophenol derivatives; benzothiazole derivatives; corticoids; tretinoin (retinoic acid; vitamin A acid); mequinol (4-hydroxyanisole); adapalene (synthetic retinoid); azelaic acid; and compounds described in WO 2012/172221; Ortonne et al., 2006.

According to the present invention, the inhibitor of the invention is administered sequentially or concomitantly with one or more anti-solar lentigo compound.

In a further aspect, the present invention relates to the inhibitor according to the invention in combination with HGF inhibitor, KGF inhibitor and SCF inhibitor for use in the treatment of solar lentigo in a subject in need thereof.

The term “HGF” has its general meaning in the art and refers to the Hepatocyte Growth Factor.

The term “HGF inhibitor” has its general meaning in the art and refers to compounds such as described in WO2009/126840; WO2009126834.

The term “KGF” has its general meaning in the art and refers to the Keratinocyte Growth Factor.

The term “KGF inhibitor” has its general meaning in the art and refers to compounds such as described in WO2006/119148; WO2001/070255; WO1994/025057.

The term “SCF” has its general meaning in the art and refers to the Stem Cell Factor.

The term “SCF inhibitor” has its general meaning in the art and refers to compounds such as described in WO2012/096960; WO2015006554.

According to the present invention, the inhibitor of the invention is administered sequentially or concomitantly with one or more HGF inhibitor, KGF inhibitor and SCF inhibitor.

In a further aspect, the present invention relates to a method of screening a candidate compound for use as a drug for the treatment of solar lentigo in a subject in need thereof, wherein the method comprises the steps of:

(i) providing an IL-13, providing IL-13 receptors, providing IL-13 downstream effectors, providing a cell, tissue sample or organism expressing the IL-13, IL-13 receptors and IL-13 downstream effectors,

(ii) providing a candidate compound such as a small organic molecule, a peptide, a polypeptide, an aptamer, an oligonucleotide, an antibody or an intra-antibodies,

(iii) measuring the IL-13 activity,

(iv) and selecting positively candidate compounds that inhibit IL-13 activity.

Methods for measuring the IL-13 activity are well known in the art (Agrawal and Townley, 2014). For example, measuring the IL-13 activity involves determining the Ki in the IL-13 cloned and transfected in a stable manner into a CHO cell line and in fibroblasts, measuring IL-13 downstream effectors activity such as JAK1/JAK2/STAT6 intracellular signaling, STAT6 phosphorylation, STAT6 nuclear translocation and TGFβ signaling in the presence or absence of the candidate compound.

Tests and assays for screening and determining whether a candidate compound is an IL-13 inhibitor are well known in the art (Agrawal and Townley, 2014). In vitro and in vivo assays may be used to assess the potency and selectivity of the candidate compounds to inhibit IL-13 activity.

Activities of the candidate compounds, their ability to bind IL-13 and their ability to inhibit IL-13 activity may be tested using isolated CHO cell line cloned and transfected in a stable manner by the human IL-13 or methods such as described in the Example.

Activities of the candidate compounds and their ability to bind to the IL-13, or their ability to inhibit IL-13 activity may be assessed by the determination of a Ki on the IL-13 cloned and transfected in a stable manner into a CHO cell line and in fibroblasts, IL-13 receptors heterodimerization, IL-13 downstream effectors signaling such as JAK1/JAK2/STAT6 intracellular signaling, STAT6 phosphorylation, STAT6 nuclear translocation and TGFβ signaling in the present or absence of the candidate compound. The ability of the candidate compounds to inhibit IL-13 activity may be assessed by measuring fibroblasts proliferation rate, migration capacity of fibroblasts by wound healing essay and migration kinetics such as described in the example.

Cells expressing another interleukin than IL-13 may be used to assess selectivity of the candidate compounds.

In another embodiment, the present invention relates to a cosmetic method for the treatment of solar lentigo in a subject in need thereof, comprising the steps of administering the IL-13 inhibitor of the invention to said subject.

In another embodiment, the present invention relates to the use of the IL-13 inhibitor of the invention for a cosmetic treatment of solar lentigo in a subject in need thereof.

In another embodiment, the present invention relates to the use of the IL-13 inhibitor of the invention for a topical cosmetic treatment of solar lentigo in a subject in need thereof.

The inhibitors of the invention may be used or prepared in a pharmaceutical composition.

In one embodiment, the invention relates to a pharmaceutical composition comprising the inhibitor of the invention and a pharmaceutical acceptable carrier for use in treating solar lentigo in a subject of need thereof.

Typically, the inhibitor of the invention may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, intramuscular, intravenous, local, topical or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, topical administration forms, granules and oral suspensions or solutions, sublingual and buccal administration forms, aerosols, implants, intraperitoneal, intramuscular, intravenous and intranasal administration forms and rectal administration forms.

Preferably, the pharmaceutical compositions contain vehicles that are pharmaceutically acceptable for a formulation capable of being injected. These may be in particular isotonic, sterile, saline solutions (monosodium or disodium phosphate, sodium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising inhibitors of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatine.

Sterile injectable solutions are prepared by incorporating the active inhibitors in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the inhibitors of the invention formulated for parenteral administration, such as intravenous or intramuscular injection, other pharmaceutically acceptable forms include, e.g. tablets or other solids for oral administration; liposomal formulations; time release capsules; and any other form currently used.

The inhibitor of the invention may be administered with a topical pharmaceutically or cosmetically acceptable carrier. The topical pharmaceutically acceptable carrier is any substantially nontoxic carrier conventionally usable for topical administration of pharmaceuticals in which the active ingredient of the invention will remain stable and bioavailable when applied directly to skin surfaces. For example, carriers such as those known in the art effective for penetrating the keratin layer of the skin into the stratum corneum may be useful in delivering the active ingredient of the invention to the area of interest. Such carriers include liposomes. Active ingredient of the invention can be dispersed or emulsified in a medium in a conventional manner to form a liquid preparation or mixed with a semi-solid (gel) or solid carrier to form a paste, powder, ointment, cream, lotion, soap, serum or the like.

Because dermatologic conditions to be treated may be visible, the topical carrier can also be a topical cosmetically acceptable carrier. The topical cosmetically acceptable carrier will be any substantially non-toxic carrier conventionally usable for topical administration of cosmetics in which the inhibitor of the invention will remain stable and bioavailable when applied directly to the skin surface. Suitable cosmetically acceptable carriers are known to those of skill in the art and include, but are not limited to, cosmetically acceptable liquids, creams, oils, lotions, ointments, gels, or solids, such as conventional cosmetic night creams, foundation creams, suntan lotions, sunscreens, hand lotions, make-up and make-up bases, masks and the like. Topical cosmetically acceptable carriers may be similar or identical in nature to the above described topical pharmaceutically acceptable carriers. The compositions can contain other ingredients conventional in cosmetics including perfumes, vitamins A, C or E, alpha-hydroxy or alpha-keto acids such as pyruvic, lactic or glycolic acids, lanolin, vaseline, aloe vera, methyl or propyl paraben, pigments and the like.

It may be desirable to have a delivery system that controls the release of the inhibitor of the invention to the skin and adheres to or maintains itself on the skin for an extended period of time to increase the contact time of the inhibitor of the invention on the skin. Sustained or delayed release of inhibitor of the invention provides a more efficient administration resulting in less frequent and/or decreased dosage of the inhibitor of the invention and better patient compliance. Examples of suitable carriers for sustained or delayed release in a moist environment include gelatin, gum arabic, xanthane polymers. Pharmaceutical carriers capable of releasing the compound of the invention when exposed to any oily, fatty, waxy, or moist environment on the area being treated, include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like. Controlled delivery systems are described, for example, in U.S. Pat. No. 5,427,778 which provides gel formulations and viscous solutions for delivery of the inhibitor of the invention to a skin site. Gels have the advantages of having a high water content to keep the skin moist, the ability to absorb skin exudate, easy application and easy removal by washing. Preferably, the sustained or delayed release carrier is a gel, liposome, microsponge or microsphere.

Compositions of the invention may include any further compound which is used in the treatment of solar lentigo.

In one embodiment, said additional active compounds may be contained in the same composition or administrated separately.

In another embodiment, the composition of the invention relates to combined preparation for simultaneous, separate or sequential use in treating solar lentigo in a subject in need thereof.

The invention also provides kits comprising the inhibitor of the invention. Kits containing the inhibitor of the invention find use in therapeutic methods and cosmetic methods.

A further aspect of the invention relates to a method of identifying a subject having or at risk of having or developing solar lentigo, comprising a step of measuring in a sample obtained from said subject the expression level of IL-13.

A further aspect of the invention relates to a method of identifying a subject having or at risk of having or developing solar lentigo, comprising a step of measuring in a sample obtained from said subject the expression level of IL-13 in combination with at least one biomarker selected from the group consisting of TGFβ1, HGF, KGF and SCF.

Typically, 2, 3, 4 or 5 biomarkers selected from the group consisting of IL-13, TGFβ1, HGF, KGF and SCF are measured.

The term “sample” refers to any sample derived from the subject such as fibroblast sample, skin biopsy sample, and hyper-pigmented skin sample.

A further aspect of the invention relates to a method of identifying a subject having or at risk of having or developing solar lentigo, comprising a step of measuring in a sample obtained from said subject the expression level of IL-13 and TGFβ1.

A further aspect of the invention relates to a method of identifying a subject having or at risk of having or developing solar lentigo, comprising a step of measuring in a sample obtained from said subject the expression level of IL-13 and HGF.

A further aspect of the invention relates to a method of identifying a subject having or at risk of having or developing solar lentigo, comprising a step of measuring in a sample obtained from said subject the expression level of IL-13 and KGF.

A further aspect of the invention relates to a method of identifying a subject having or at risk of having or developing solar lentigo, comprising a step of measuring in a sample obtained from said subject the expression level of IL-13 and SCF.

A further aspect of the invention relates to a method of identifying a subject having or at risk of having or developing solar lentigo, comprising a step of measuring in a sample obtained from said subject the expression level of IL-13, TGFβ1 and HGF.

A further aspect of the invention relates to a method of identifying a subject having or at risk of having or developing solar lentigo, comprising a step of measuring in a sample obtained from said subject the expression level of IL-13, TGFβ1 and KGF.

A further aspect of the invention relates to a method of identifying a subject having or at risk of having or developing solar lentigo, comprising a step of measuring in a sample obtained from said subject the expression level of IL-13, TGFβ1 and SCF.

A further aspect of the invention relates to a method of identifying a subject having or at risk of having or developing solar lentigo, comprising a step of measuring in a sample obtained from said subject the expression level of IL-13, HGF and KGF.

A further aspect of the invention relates to a method of identifying a subject having or at risk of having or developing solar lentigo, comprising a step of measuring in a sample obtained from said subject the expression level of IL-13, HGF and SCF.

A further aspect of the invention relates to a method of identifying a subject having or at risk of having or developing solar lentigo, comprising a step of measuring in a sample obtained from said subject the expression level of IL-13, KGF and SCF.

A further aspect of the invention relates to a method of identifying a subject having or at risk of having or developing solar lentigo, comprising a step of measuring in a sample obtained from said subject the expression level of IL-13, TGFβ1, HGF and KGF.

A further aspect of the invention relates to a method of identifying a subject having or at risk of having or developing solar lentigo, comprising a step of measuring in a sample obtained from said subject the expression level of IL-13, TGFβ1, HGF and SCF.

A further aspect of the invention relates to a method of identifying a subject having or at risk of having or developing solar lentigo, comprising a step of measuring in a sample obtained from said subject the expression level of IL-13, TGFβ1, KGF and SCF.

A further aspect of the invention relates to a method of identifying a subject having or at risk of having or developing solar lentigo, comprising a step of measuring in a sample obtained from said subject the expression level of IL-13, HGF, KGF and SCF.

A further aspect of the invention relates to a method of identifying a subject having or at risk of having or developing solar lentigo, comprising a step of measuring in a sample obtained from said subject the expression level of IL-13, TGFβ1, HGF, KGF and SCF.

The method of the invention may further comprise a step consisting of comparing the expression level of the biomarkers in the sample with a reference value, wherein detecting differential in the expression level of the biomarkers between the sample and the reference value is indicative of subject having or at risk of having or developing solar lentigo.

As used herein, the “reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the expression level (obtained according to the method of the invention) with a defined threshold value. In one embodiment of the present invention, the threshold value is derived from the expression level (or ratio, or score) determined in a sample derived from one or more subjects having solar lentigo. Furthermore, retrospective measurement of the expression level (or ratio, or scores) in properly banked historical subject samples may be used in establishing these threshold values.

In one embodiment, the reference value may correspond to the expression level of the biomarker determined in a sample associated with a subject not afflicted with solar lentigo or a non-lesional biopsy such as described in the example. Accordingly, a higher expression level of the biomarker than the reference value is indicative of a subject having or at risk of having or developing solar lentigo, and a lower or equal expression level of the biomarker than the reference value is indicative of a subject not having or not at risk of having or developing solar lentigo.

In another embodiment, the reference value may correspond to the expression level of the biomarker determined in a sample associated with a subject afflicted with solar lentigo. Accordingly, a higher or equal expression level of the biomarker than the reference value is indicative of a subject having or at risk of having or developing solar lentigo, and a lower expression level of the biomarker than the reference value is indicative of a subject not having or not at risk of having or developing solar lentigo.

In another embodiment, a score which is a composite of the expression levels of the different biomarkers may also be determined and compared to a reference value wherein a difference between said score and said reference value is indicative of a subject having or at risk of having or developing solar lentigo

In a particular embodiment, the score may be generated by a computer program.

Analyzing the biomarker expression level may be assessed by any of a wide variety of well-known methods for detecting expression of a transcribed nucleic acid or translated protein.

In one embodiment, the biomarker expression level is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for the biomarkers.

Methods for measuring the expression level of a biomarker in a sample may be assessed by any of a wide variety of well-known methods from one of skill in the art for detecting expression of a protein including, but not limited to, direct methods like mass spectrometry-based quantification methods, protein microarray methods, enzyme immunoassay (EIA), radioimmunoassay (RIA), Immunohistochemistry (IHC), Western blot analysis, ELISA, Luminex, ELISPOT and enzyme linked immunoabsorbant assay and undirect methods based on detecting expression of corresponding messenger ribonucleic acids (mRNAs). The mRNA expression profile may be determined by any technology known by a man skilled in the art. In particular, each mRNA expression level may be measured using any technology known by a man skilled in the art, including nucleic microarrays, quantitative Polymerase Chain Reaction (qPCR), next generation sequencing and hybridization with a labelled probe.

Said direct analysis can be assessed by contacting the sample with a binding partner capable of selectively interacting with the biomarker present in the sample. The binding partner may be an antibody that may be polyclonal or monoclonal, preferably monoclonal (e.g., a isotope-label, element-label, radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for the biomarkers of the invention. In another embodiment, the binding partner may be an aptamer.

The binding partners of the invention such as antibodies or aptamers, may be labelled with a detectable molecule or substance, such as an isotope, an element, a fluorescent molecule, a radioactive molecule or any others labels known in the art. Labels are known in the art that generally provide (either directly or indirectly) a signal.

As used herein, the term “labelled”, with regard to the antibody, is intended to encompass direct labelling of the antibody or aptamer by coupling (i.e., physically linking) a detectable substance, such as an isotope, an element, a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be produced with a specific isotope or a radioactive molecule by any method known in the art. For example radioactive molecules include but are not limited to radioactive atom for scintigraphic studies such as 1123, 1124, In111, Re186, Re188, specific isotopes include but are not limited to 13C, 15N, 126I, 79Br, 81Br.

The afore mentioned assays generally involve the binding of the binding partner (ie. antibody or aptamer) to a solid support. Solid supports which can be used in the practice of the invention include substrates such as nitrocellulose (e. g., in membrane or microtiter well form);

polyvinylchloride (e. g., sheets or microtiter wells); polystyrene latex (e.g., beads or microtiter plates); polyvinylidene fluoride; diazotized paper; nylon membranes; activated beads, magnetically responsive beads, silicon wafers.

In a particular embodiment, an ELISA method can be used, wherein the wells of a microtiter plate are coated with a set of antibodies which recognize said biomarker(s). A sample containing or suspected of containing said biomarker(s) is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art such as Singulex, Quanterix, MSD, Bioscale, Cytof.

In one embodiment, an Enzyme-linked immunospot (ELISpot) method may be used. Typically, the sample is transferred to a plate which has been coated with the desired anti-biomarker capture antibodies. Revelation is carried out with biotinylated secondary Abs and standard colorimetric or fluorimetric detection methods such as streptavidin-alkaline phosphatase and NBT-BCIP and the spots counted.

In one embodiment, when multi-biomarker expression measurement is required, use of beads bearing binding partners of interest may be preferred. In a particular embodiment, the bead may be a cytometric bead for use in flow cytometry. Such beads may for example correspond to BD™ Cytometric Beads commercialized by BD Biosciences (San Jose, Calif.). Typically cytometric beads may be suitable for preparing a multiplexed bead assay. A multiplexed bead assay, such as, for example, the BD™ Cytometric Bead Array, is a series of spectrally discrete beads that can be used to capture and quantify soluble antigens. Typically, beads are labelled with one or more spectrally distinct fluorescent dyes, and detection is carried out using a multiplicity of photodetectors, one for each distinct dye to be detected. A number of methods of making and using sets of distinguishable beads have been described in the literature. These include beads distinguishable by size, wherein each size bead is coated with a different target-specific antibody (see e.g. Fulwyler and McHugh, 1990, Methods in Cell Biology 33:613-629), beads with two or more fluorescent dyes at varying concentrations, wherein the beads are identified by the levels of fluorescence dyes (see e.g. European Patent No. 0 126,450), and beads distinguishably labelled with two different dyes, wherein the beads are identified by separately measuring the fluorescence intensity of each of the dyes (see e.g. U.S. Pat. Nos. 4,499,052 and 4,717,655). Both one-dimensional and two-dimensional arrays for the simultaneous analysis of multiple antigens by flow cytometry are available commercially. Examples of one-dimensional arrays of singly dyed beads distinguishable by the level of fluorescence intensity include the BD™ Cytometric Bead Array (CBA) (BD Biosciences, San Jose, Calif.) and Cyto-Plex™ Flow Cytometry microspheres (Duke Scientific, Palo Alto, Calif.). An example of a two-dimensional array of beads distinguishable by a combination of fluorescence intensity (five levels) and size (two sizes) is the QuantumPlex™ microspheres (Bangs Laboratories, Fisher, Ind.). An example of a two-dimensional array of doubly-dyed beads distinguishable by the levels of fluorescence of each of the two dyes is described in Fulton et al. (1997, Clinical Chemistry 43(9):1749-1756). The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange). In another particular embodiment, bead is a magnetic bead for use in magnetic separation. Magnetic beads are known to those of skill in the art. Typically, the magnetic bead is preferably made of a magnetic material selected from the group consisting of metals (e.g. ferrum, cobalt and nickel), an alloy thereof and an oxide thereof. In another particular embodiment, bead is bead that is dyed and magnetized.

In one embodiment, protein microarray methods may be used. Typically, at least one antibody or aptamer directed against the biomarker(s) is immobilized or grafted to an array(s), a solid or semi-solid surface(s). A sample containing or suspected of containing the biomarker(s) is then labelled with at least one isotope or one element or one fluorophore or one colorimetric tag that are not naturally contained in the tested sample. After a period of incubation of said sample with the array sufficient to allow the formation of antibody-antigen complexes, the array is then washed and dried. After all, quantifying said biomarkers may be achieved using any appropriate microarray scanner like fluorescence scanner, colorimetric scanner, SIMS (secondary ions mass spectrometry) scanner, maldi scanner, electromagnetic scanner or any technique allowing to quantify said labels.

In another embodiment, the antibody or aptamer grafted on the array is labelled.

In another embodiment, reverse phase arrays may be used. Typically, at least one sample is immobilized or grafted to an array(s), a solid or semi-solid surface(s). An antibody or aptamer against the suspected biomarker(s) is then labelled with at least one isotope or one element or one fluorophore or one colorimetric tag that are not naturally contained in the tested sample. After a period of incubation of said antibody or aptamer with the array sufficient to allow the formation of antibody-antigen complexes, the array is then washed and dried. After all, detecting quantifying and counting by D-SIMS said biomarkers containing said isotope or group of isotopes, and a reference natural element, and then calculating the isotopic ratio between the biomarkers and the reference natural element. may be achieve using any appropriate microarray scanner like fluorescence scanner, colorimetric scanner, SIMS (secondary ions mass spectrometry) scanner, maldi scanner, electromagnetic scanner or any technique allowing to quantify said labels.

In one embodiment, said direct analysis can also be assessed by mass Spectrometry. Mass spectrometry-based quantification methods may be performed using either labelled or unlabeled approaches (DeSouza and Siu, 2012). Mass spectrometry-based quantification methods may be performed using chemical labeling, metabolic labeling or proteolytic labeling. Mass spectrometry-based quantification methods may be performed using mass spectrometry label free quantification, LTQ Orbitrap Velos, LTQ-MS/MS, a quantification based on extracted ion chromatogram EIC (progenesis LC-MS, Liquid chromatography-mass spectrometry) and then profile alignment to determine differential expression of biomarkers.

In another embodiment, the biomarkers expression level is assessed by analyzing the expression of mRNA transcript or mRNA precursors, such as nascent RNA, of biomarkers gene. Said analysis can be assessed by preparing mRNA/cDNA from cells in a sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFFYMETRIX).

Advantageously, the analysis of the expression level of mRNA transcribed from the gene encoding for biomarkers involves the process of nucleic acid amplification, e. g., by RT-PCR (the experimental embodiment set forth in U.S. Pat. No. 4,683, 202), ligase chain reaction (Barany, 1991), self sustained sequence replication (Guatelli et al., 1990), transcriptional amplification system (Kwoh et al., 1989), Q-Beta Replicase (Lizardi et al., 1988), rolling circle replication (U.S. Pat. No. 5,854, 033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In a further aspect, the method of the invention is performed by measuring IL-13 activation level.

Analyzing the IL-13 activation level may be assessed by any of a wide variety of well-known methods such as measuring IL-13 receptors heterodimerization, IL-13 downstream effectors signaling such as JAK1/JAK2/STAT6 intracellular signaling, STAT6 phosphorylation, STAT6 nuclear translocation and TGFβ signaling.

A further aspect of the invention relates to a method of monitoring solar lentigo progression by performing the method of the invention.

In one embodiment, the present invention relates to a method of treating solar lentigo in a subject in need thereof comprising the steps of:

(i) identifying a subject having or at risk of having or developing a solar lentigo by performing the method according to the invention, and

(ii) administering to said subject an IL-13 inhibitor compound.

The invention will be further illustrated by the following figures and examples. However, these examples and figures should not be interpreted in any way as limiting the scope of the present invention.

### FIGURES

FIG. 1: Differential cellular features between adjacent- and solar lentigo-isolated fibroblasts. Heterogeneous morphologies of fibroblasts isolated from peri-lesional (FS) and solar lentigo (FL) skin biopsies after examination by phase contrast microscopy (n=10 FS/FL couples) and representative immuno fluorescence images of FS and FL fibroblasts (n=10) showing F-actin labelling and Hoechst (nuclei) was performed. (a) F-actin staining was quantified in both FS (n=119) and FL (n=102) and graphed. Based on elliptical form parameter, quantitative analyses of cellular body (b) and nucleus (c) shapes of both FS and FL fibroblasts are shown; ***p<0.0001.

FIG. 2: Differential proliferation rates and migration capacities between FL and FS. Equal number of FS and FL were seeded at time 0. Proliferation rate was determined after counting cells 96 hours later (a; “p<0.002; n=10) and metabolic activity was assessed using MTT assay after 24 hours (b; **p<0.003; n=10). FS and FL migration capacity was determined after a wound healing assay (c). Percentage of wound closure was calculated for each time point and average of percentages in 3 FS/FL samples were plotted; ****p<0.0001.

FIG. 3: Differential secretion profiles for HGF, KGF, SCF, IL-13 and TGFβ1 between FL and FS. FL- and FS-conditioned media (n=10 couples) were subjected to quantification for 3 growth factors using ELISA (a) and 31 other soluble factors using V-Plex (b). After normalization to FS or FL number, concentration of these factors was determined and graphed (HGF ***p<0.0005, KGF *p<0.05, SCF *p<0.05, IL-13 *p<0.0294 and TGFβ1 **p<0.0076).

FIG. 4: α-SMA staining intensity is similar between FS and FL. Immunofluorescence images of FS and FL fibroblasts (n=10) showing α-SMA labelling and Hoechst (nuclei) was performed. α-SMA staining was quantified in both FS (n=119) and FL (n=102) and graphed. ns: non significant.

FIG. 5: Differential morphological and functional features between non lesional- and solar lentigo-explanted fibroblasts. Images of representative senescence-associated β-galactosidase (SA-β-Gal) staining of 2 FNL/FL couples and normal fibroblasts subjected (FUV) or not (FNUV) to repeated-UVB exposures. Quantification of β-Gal positive fibroblasts showed that FL (n=87/388) were more senescent than FNL cells (n=27/514) (**** p<0.0001); FNUV (n=5/458) and FUV (n=269/353) were used as controls.

### EXAMPLES

### Example 1

Morphological and Functional Characterizations of Fibroblasts Extracted from Solar Lentigo

Material & Methods

Phosphate buffer solution (PBS), Dulbecco's modified Eagle's medium (DMEM), fetal calf serum (FCS), gentamycin, trypsin and all cell culture plastics were purchased from Dutscher (Brumath, France); paraformaldehyde (PFA), 3-(4,5-dimethylthiazol-2-yl)-2,5-diphenyltetrazolium bromide (MTT), sodium dodecyl sulfate (SDS), dimethylformamide (DMF), glycine, acetone, triton X-100, bovine serum albumin (BSA), mouse α-SM actin and goat anti-mouse IgG-FITC antibodies, rhodamine-conjugated phalloidin, Hoechst were purchased from Sigma-Aldrich (St Quentin Fallavier, France).

Human Skin Biopsies

Skin specimens were obtained from 13 volunteers on the back of the hands (Caucasian females, 67-89 years of age) after written informed consent (Pr. P. Humbert, University hospital of Besancon, France). Two biopsies of 3-mm punches were collected from the same patient with one solar lentigo lesional skin and one non-pigmented peri-lesional skin.

Histological Detection of Melanin

Briefly, Solar Lentigo (SL) and Peri-lesional (PL) biopsies were fixed in 3% PFA solution, dehydrated and embedded in paraffin. Seven-μm thick sections were cut using a rotary microtome (Leitz 1512; Leica) and stained with Fontana-Masson following the manufacturer's instructions (Diapath). After mounting, histological features of the skin sections were examined by optical microscopy (Axioskop 40, Zeiss).

Cell Cultures

Primary culture of human dermal fibroblasts obtained from SL (FL) and PL (FS) of 10 patients were maintained in DMEM supplemented with 10% FCS and 1% Gentamycin in a humidified 5% CO2 atmosphere at 37° C. FL and FS cells were maintained until the monolayer cultures reached 80% confluency and were passaged twice using a trypsin (0.05%)/EDTA (0.02%) solution.

Cell Proliferation

Proliferation of FL and FS was determined by measuring metabolic activity (colorimetric MTT assay) 30 and by counting viable cells (Trypan blue exclusion assay).

MTT Assay

FL and FS were seeded into 96-well plates (5×103 cells/well) and incubated with DMEM supplemented for 24 h. After removing supernatant and washing by PBS 1×, 10 μl of MTT solution (0.5 mg/mL in DMEM) were added for 4 h at 37° C. The formazan-blue crystals were dissolved overnight at 37° C. in 100 μL of extraction medium (10% SDS/0.04% DMF in H2O). The optical density was read at 570 nm with a Multiskan FCTM spectrophotometer

(Fisher Scientific).

Trypan Blue Exclusion Assay

FL and FS were seeded at a density of 7×105 cells/T75 flask and were incubated with DMEM supplemented for 4 days. Cells were trypsinized and count by Trypan blue stain.

Fluorescence Staining

Primary FL and FS were seeded in Lab-Tek chamber slides at 1×104 cells/chamber and maintained in culture at 37° C. for 24 h. They were then fixed 10 min in 3% PFA solution and permeabilized for 10 min with cold acetone at −20° C. Nonspecific adhesion sites were blocked by a 10 min incubation in 1% glycine solution followed by a 1 h incubation in BSA solution (3% BSA/10% serum/0.1% triton X-100 in PBS). Primary antibody directed against human α-SMA was then added (1: 100 in PBS/1%BSA/0.1% triton X-100) and incubated overnight at 4° C. in a moist chamber. Cells were then incubated with the FITC conjugated secondary antibody (1: 40 in PBS) for 1 h and further 30 min with rhodamine-conjugated phalloidin for filamentous actin (F-actin) staining (2.5 μg/ml). Cellular nuclei were countermarked with Hoechst solution. Cells were then mounted in Dako fluorescent medium, observed under fluorescence microscopy (Axioskop 40, Zeiss) and further analyzed using Metamorph software (Molecular Devices).

Wound Healing Assay

Primary FL and FS were grown on 12-well Falcon® plates for 24 h to 48 h to reach 100% cell confluent monolayers. Wounds were done using a sterile 10 μl-pipette tip across each well. After gently washes, cells were incubated with supplemented DMEM w/o Red phenol, covered with mineral oil and placed in the incubator at 37° C. with 5% CO2 of the BD Pathway 855 Bioimaging (Becton Dickinson, USA). Image captures at 1 h-regular intervals using the AttoVision Software (Becton Dickinson) allowed calculating the percentage of wound closure for each time point (ImageJ Software).

ELISA and V-Plex Assays

Secreted factors in paired FL/FS-conditioned media were quantified by ELISA kits (Quantikine® for KGF, HGF, SCF and TGF-β1; R&D Systems) or by MSD 30-PLEX™ Proinflammatory Panell, Cytokine Panell, Chemokine Panell kits and TGFβ1 kit, according to the manufacture's protocols.

Statistical Analysis

Statistical significance was assessed using paired Student's t test (Prism5.0, Graph Pad Software). P values were considered significant with the following degrees: *p<0.05; **p<0.01; ***p<0.001.

Results

Melanin Staining of Human SL and Peri-Lesional Skin Biopsies

To check that human skin fibroblasts used in this study were isolated from peri-lesional and lesional skins, biopsies from 3 volunteers who presented solar lentigo were subjected to Fontana-Masson staining. Peri-lesional (PL) and lesional (SL) tissues displayed different histological features. SL showed a thin epidermis, disorganized basement membrane, a high melanin staining of the basal layer, intense melanin deposition and elongated rete ridges collapsing into the dermis. Altogether, microscopic analysis validated the SL origin of our in vitro fibroblastic model.

Differential Morphological Features Between Isolated Fibroblasts from Peri-Lesional and SL Skin Biopsies

Heterogeneous morphologies of fibroblasts isolated from peri-lesional (FS) and solar lentigo (FL) skin biopsies after examination by phase contrast microscopy (n=10 FS/FL couples) and immunofluorescence images of FS and FL fibroblasts (n=10) showing F-actin labelling and Hoechst (nuclei) was performed (data not shown).

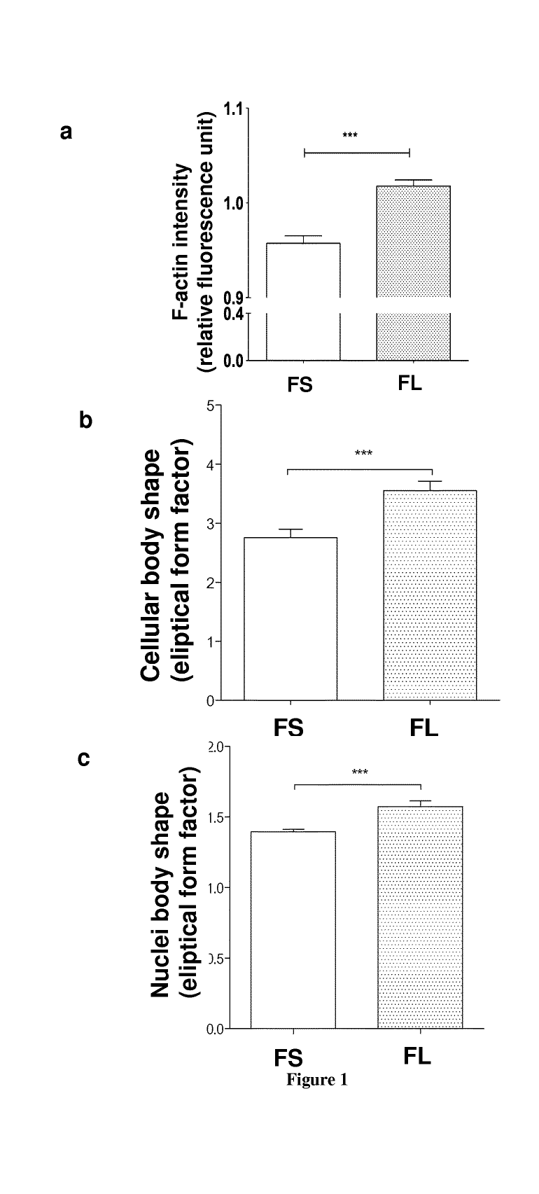

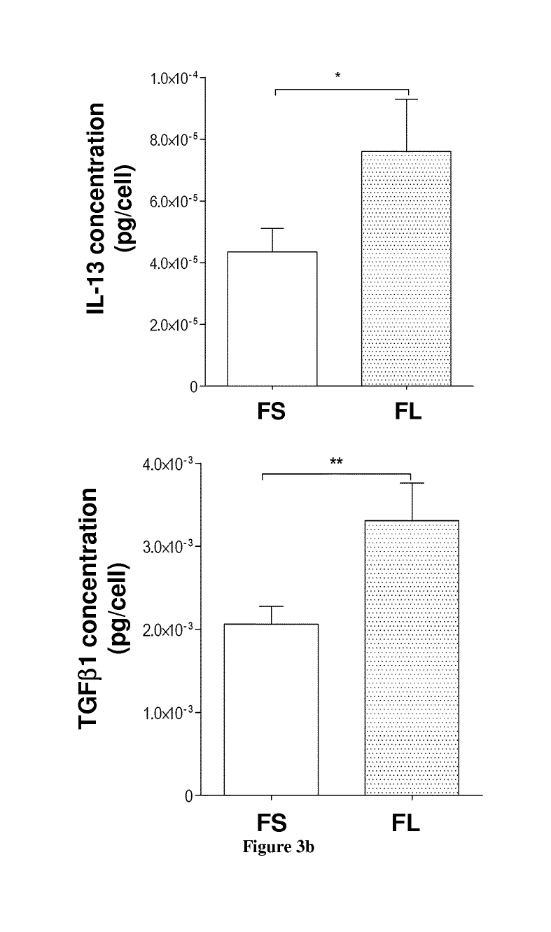

Then, we undertook the morphological characterization of the fibroblasts isolated from non-lesional (FS) and solar lentigo (FL) skin biopsies. Global microscopic examination revealed that both types of primary fibroblasts exhibited variable shapes and sizes (data not shown). Fluorescent phalloidin staining showed that FL exhibited a more heterogeneous network of F-actin with a much denser labelling of stress fibers (data not shown). Quantification of the F-actin intensity staining in FS (n=119) and FL (n=102) revealed that FL staining level was significantly higher relative to FS (FIG. 1a). Moreover, analysis of the cellular body and nuclear appearances showed that FL displayed more elongated shapes (FIG. 1b) with an elliptical shaped nucleus (FIG. 1c) as compared with FS. In contrast, immunofluorescence analysis for the α-SMA, a hallmark of the myofibroblastic phenotype, did not show difference in its intensity between FS and FL (FIG. 4).

Altogether, these data demonstrate distinct morphological characteristics between both fibroblastic cell types without any difference in their myofibroblastic feature.

Differential Functional Characteristics Between Isolated Fibroblasts from Peri-Lesional and SL Skin Biopsies

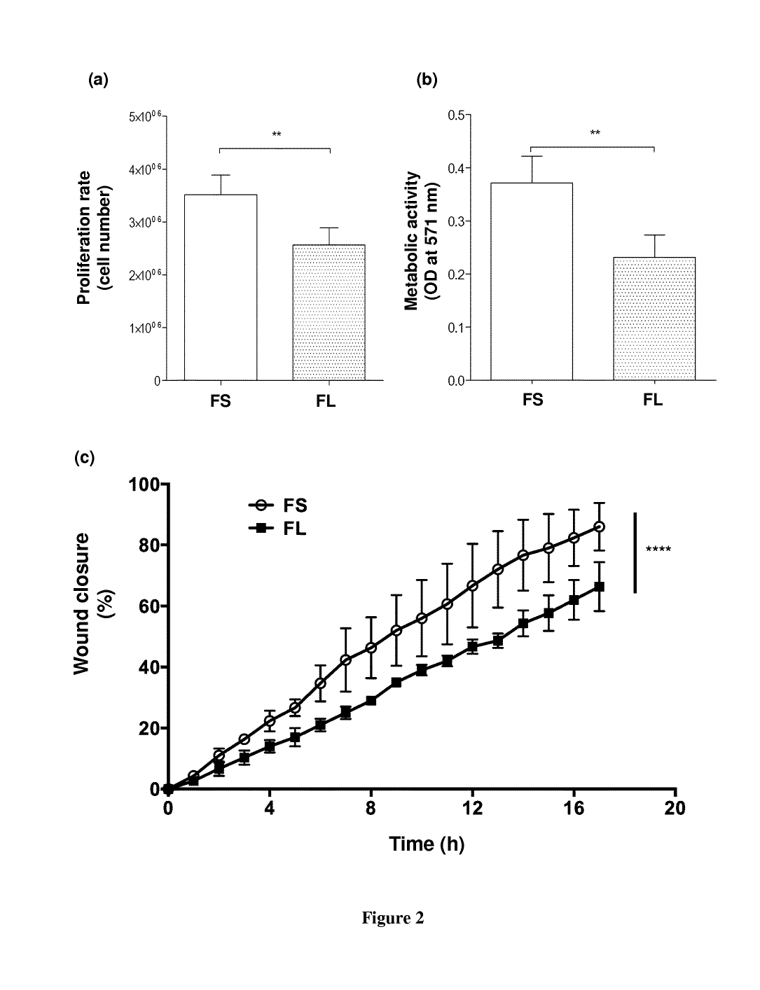

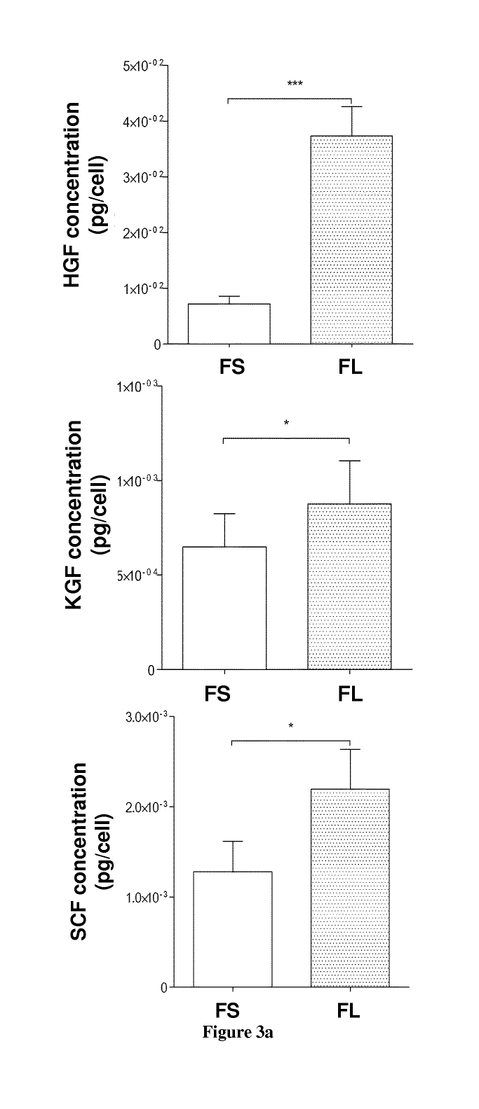

To functionally explore both types of fibroblasts, we first analysed their proliferation rate by seeding equal number of FS and FL and counting them at 96 h of culture. As shown in the FIG. 2a, cell number was significantly lower in FL relative to FS. Accordingly, measurement of metabolically intact cells demonstrated that FL proliferation was significantly reduced as compared to FS (FIG. 2b). Next, we examined the migration capacity of both fibroblastic types by wound healing assay and calculated the percentage of wound closure for each time point. Results presented in the FIG. 2c demonstrated that migration kinetics of FL were significantly lower relative to FS. Finally, secretion capacity of both types of fibroblasts was determined by quantifying cytokines and growth factors present in their respective conditioned media by ELISA and V-plex assays. As shown in FIG. 3a, HGF, KGF and SCF were detected in both FS and FL but significantly at higher rates in FL. We further investigated the secretion pattern of 31 additional soluble factors that were chosen based on their role in inflammation. Interestingly, the cytokine IL-13 and the growth factor TGFβ1 were significantly increased in the FL media as compared to FS (FIG. 3b); the concentration of the 29 other soluble factors was neither differentially secreted (Eotaxin, Eotaxin-3, GM-CSF, IFN-γ, IL-10, IL-12p70, IL-16, IL-17A, IL-4, IL-6, IL-7, IL-8/IL-8 (HA), IP-10, MCP-4, MDC, MIP-1α, MIP-1β, TARC, TNF-α, VEGF-A) nor detectable (IL-12/IL-23p40, IL-15, IL-1α, IL-1α, IL-2, IL-5, IL8 cck, MCP-1, TNF-β).

Collectively, functional study of the fibroblasts isolated from SL demonstrated that their proliferation rate, metabolic activity and migration capacity were lower as compared to their intra-individual counterparts whereas their secretion profiles were higher for some soluble factors that are involved in tissue fibrosis.

Discussion

To our knowledge, this study constitutes the first description of the isolation of primary fibroblasts from peri-lesional and SL biopsies that were issued and compared from the same volunteer. By staining human peri-lesional and SL skin samples for melanin, we validated the origin of the FS and FL and identified some of their morphological and functional features.

It is now accepted that fibroblasts represent a heterogeneous population of cells found in most of the tissues (6, 7). Accordingly, our microscopic observations revealed that FS and FL exhibit heterogeneous shapes and sizes with FL characterized by more elongated shapes and elliptical shaped nuclei. Given that actin isoforms are markers of the fibroblast heterogeneity with their differential expression and organization of cytoskeletal proteins (31, 32), we looked at the F-actin iso form. Spread FS and FL showed bundles that were quite similar but not identical in terms of cables length, number and distribution. Indeed, FL showed noticeable actin stress fibers in their cytoplasm that were linked to higher quantity of F-actin, suggesting that both types of fibroblasts have different actin-based cytoskeletal frameworks. Looking at the α-SM actin isoform, similar quantities of this myofibroblast marker were found in FS and FL. This observation was in agreement with the absence of difference between peri-lesional and SL skin in terms of α-SMA-positive cells in the dermis (33). Despite evidence describing that fibroblasts isolated from tissues continually “differentiate” into myofibroblast-like cells with different degrees of α-SMA expression once they are grown in culture (34, 35), its is tempting to speculate that our primary SL-fibroblast model behaves, in terms of myofibroblast differentiation, like the fibroblasts described in SL biopsies.

Functional studies demonstrated that both fibroblasts displayed differential proliferation rate with a lower level for FL. Apparent conflicting immunohistochemistry data, generated with the proliferation marker Ki67, were brought back together by demonstrating that expression of this marker is modulated during SL development. Indeed, Ki67-positive cells are strongly reduced at the later stages relative to early stages (23). Despite the association between Ki67-positive cells and supra-basal keratinocytes in tissue sections (23) and, given that our SL samples were probably of the later SL stages, our results strongly suggested that fibroblasts contribute to the cellular quiescent status observed in advanced SL.

Another biological function of the fibroblast consists on its migration capacity that is related to actin cytoskeleton, integrin adhesion molecules and extracellular matrix proteins (36, 37). According to the different cell morphology and cytoplasmic structural organization between FL and FS, cell mobility was also different between FS and FL. The decrease of FL motility relative to FS could be explained by impaired integrin or extracellular matrix protein expression.

The last function studied was the secretion profile of FS and FL, which contributes to cellular functional crosstalks. Indeed, melanocytes and their neighbouring keratinocytes regulate mutually their functions through networks of factors (10, 27, 38, 39). Moreover, recent evidence points out functional implication of dermal fibroblast secretion in modulating not only constitutive pigmentation (10, 11, 40-42) but also the development of various hyper-pigmented disorders, via the secretion of growth factors such as SCF (43-45), HGF (43, 45) and KGF (46). In this context, we first quantified these melanogenic factors in our primary fibroblastic models that were issued from peri-lesional and SL biopsies. Higher secretion levels of SCF, HGF and KGF detected in FL media supported published data showing higher immuno-detection levels of these 3 growth factors in the upper dermis of SL skin (28). This strongly suggests that the secretion capacity of our in vitro primary fibroblast model was close to fibroblasts located in the upper dermis of SL.

In line with the chronic inflammatory context of the SL (8, 25, 26), our findings also demonstrated that TGFβ1 and IL-13 (47, 48) were differentially secreted between FS and FL with a higher level for FL. While the ubiquitous TGFβ1 has been reported to inhibit in vivo keratinocyte proliferation (47, 49) and in vitro melanocyte differentiation 2, the endogenous secretion of the cytokine IL-13 by dermal fibroblasts, melanocytes and keratinocytes has never been described. Nevertheless, further mechanism studies using neutralizing antibodies should clarify the role of these soluble factors in SL progression.

In conclusion, isolation of fibroblasts from adjacent- and SL-skin biopsies allowed establishing primary dermal cell models with some specific morphological and functional features that are similar to those described in SL biopsies. By identifying FS and FL secretion signatures, new investigations are required to clarify the autocrine/paracrine roles of the differential concentrations of soluble factors on melanocyte and keratinocyte functions. It is tempting to speculate that our primary fibroblast model might constitute a good cellular tool to test compounds for the development of whitening and anti-aging agents in topical treatments.

### REFERENCES

Throughout this application, various references describe the state of the art to which this invention pertains. The disclosures of these references are hereby incorporated by reference into the present disclosure.

1. Yamaguchi, Y. & Hearing, V. J. Physiological factors that regulate skin pigmentation. Biofactors 35, 193-9 (2009).

2. Slominski, A., Tobin, D. J., Shibahara, S. & Wortsman, J. Melanin pigmentation in mammalian skin and its hormonal regulation. Physiol Rev 84, 1155-228 (2004).

3. Matsui, T. & Amagai, M. Dissecting the formation, structure and barrier function of the stratum corneum. Int Immuno127, 269-280 (2015).

4. Haniffa, M., Gunawan, M. & Jardine, L. Human skin dendritic cells in health and disease. J Dermatol Sci 77, 85-92 (2015).

5. Woo, S. H., Lumpkin, E. A. & Patapoutian, A. Merkel cells and neurons keep in touch. Trends Cell Biol 25, 74-81 (2015).

6. Driskell, R. R. & Watt, F. M. Understanding fibroblast heterogeneity in the skin. Trends Cell Biol 25, 92-9 (2015).

7. Sorrell, J. M. & Caplan, A. I. Fibroblasts-a diverse population at the center of it all. Int Rev Cell Mol Biol 276, 161-214 (2009).

8. Cardinali, G., Kovacs, D. & Picardo, M. [Mechanisms underlying post-inflammatory hyperpigmentation: lessons from solar lentigo]. Ann Dermatol Venereol 139 Suppl 3, S96-101 (2012).

9. Kondo, T. & Hearing, V. J. Update on the regulation of mammalian melanocyte function and skin pigmentation. Expert Rev Dermatol 6, 97-108 (2011).

10. Duval, C. et al. Key regulatory role of dermal fibroblasts in pigmentation as demonstrated using a reconstructed skin model: impact of photo-aging. PLoS One 9, e114182 (2014).

11. Hedley, S. J. et al. Fibroblasts play a regulatory role in the control of pigmentation in reconstructed human skin from skin types I and II. Pigment Cell Res 15, 49-56 (2002).

12. Cario-Andre, M., Pain, C., Gauthier, Y., Casoli, V. & Taieb, A. In vivo and in vitro evidence of dermal fibroblasts influence on human epidermal pigmentation. Pigment Cell Res 19, 434-42 (2006).

13. Yang, R. et al. Direct conversion of mouse and human fibroblasts to functional melanocytes by defined factors. Nat Commun 5, 5807 (2014).

14. Praetorius, C., Sturm, R. A. & Steingrimsson, E. Sun-induced freckling: ephelides and solar lentigines. Pigment Cell Melanoma Res 27, 339-50 (2014).

15. Cayce, K. A., McMichael, A. J. & Feldman, S. R. Hyperpigmentation: an overview of the common afflictions. Dermatol Nurs 16, 401-6, 413-6; quiz 417 (2004).

16. Bastiaens, M., Hoelhagel, J., Westendorp, R., Vermeer, B. J. & Bouwes Bavinck, J. N. Solar lentigines are strongly related to sun exposure in contrast to ephelides. Pigment Cell Res 17, 225-9 (2004).

17. Nouveau-Richard, S. et al. Skin aging: a comparison between Chinese and European populations. A pilot study. J Dermatol Sci 40, 187-93 (2005).

18. Monestier, S., Gaudy, C., Gouvernet, J., Richard, M. A. & Grob, J. J. Multiple senile lentigos of the face, a skin aging pattern resulting from a life excess of intermittent sun exposure in dark-skinned caucasians: a case-control study. Br J Dermatol 154, 438-44 (2006).

19. Ortonne, J. P. & Bissett, D. L. Latest insights into skin hyperpigmentation. J Investig Dermatol Symp Proc 13, 10-4 (2008).

20. Nakamura, M. et al. Environment-induced lentigines: formation of solar lentigines beyond ultraviolet radiation. Exp Dermatol 24, 407-11 (2015).

21. Andersen, W. K., Labadie, R. R. & Bhawan, J. Histopathology of solar lentigines of the face: a quantitative study. J Am Acad Dermatol 36, 444-7 (1997).

22. Unver, N. et al. Alterations in the epidermal-dermal melanin axis and factor XIIIa melanophages in senile lentigo and aging skin. Br J Dermatol 155, 119-28 (2006).

23. Lin, C. B. et al. Immuno-histochemical evaluation of solar lentigines: The association of KGF/KGFR and other factors with lesion development. J Dermatol Sci 59, 91-7 (2010).

24. Goorochurn, R. et al. Biological processes in solar lentigo: insights brought by experimental models. Exp Dermatol (2016).

25. Aoki, H., Moro, O., Tagami, H. & Kishimoto, J. Gene expression profiling analysis of solar lentigo in relation to immunohistochemical characteristics. Br J Dermatol 156, 1214-23 (2007).

26. Goyarts, E., Muizzuddin, N., Maes, D. & Giacomoni, P. U. Morphological changes associated with aging: age spots and the microinflammatory model of skin aging. Ann N Y Acad Sci 1119, 32-9 (2007).

27. Imokawa, G. Autocrine and paracrine regulation of melanocytes in human skin and in pigmentary disorders. Pigment Cell Res 17, 96-110 (2004).

28. Kovacs, D. et al. Role of fibroblast-derived growth factors in regulating hyperpigmentation of solar lentigo. Br J Dermatol 163, 1020-7 (2010).

29. Kim, M., Han, J. H., Kim, J. H., Park, T. J. & Kang, H. Y. Secreted Frizzled-Related Protein 2 (sFRP2) Functions as a Melanogenic Stimulator; The Role of sFRP2 in UV-Induced Hyperpigmentary Disorders. J Invest Dermatol (2015).

30. Mosmann, T. Rapid colorimetric assay for cellular growth and survival: application to proliferation and cytotoxicity assays. J Immunol Methods 65, 55-63 (1983).

31. Sappino, A. P., Schurch, W. & Gabbiani, G. Differentiation repertoire of fibroblastic cells: expression of cytoskeletal proteins as marker of phenotypic modulations. Lab Invest 63, 144-61 (1990).

32. Gardel, M. L., Schneider, I. C., Aratyn-Schaus, Y. & Waterman, C. M. Mechanical integration of actin and adhesion dynamics in cell migration. Annu Rev Cell Dev Bio126, 315-33 (2010).

33. Cario-Andre, M. et al. Perilesional vs. lesional skin changes in senile lentigo. J Cutan Pathol 31, 441-7 (2004).

34. Dugina, V., Alexandrova, A., Chaponnier, C., Vasiliev, J. & Gabbiani, G. Rat fibroblasts cultured from various organs exhibit differences in alpha-smooth muscle actin expression, cytoskeletal pattern, and adhesive structure organization. Exp Cell Res 238, 481-90 (1998).

35. Hinz, B., Celetta, G., Tomasek, J. J., Gabbiani, G. & Chaponnier, C. Alpha-smooth muscle actin expression upregulates fibroblast contractile activity. Mol Biol Cell 12, 2730-41 (2001).

36. Li, W., Fan, J., Chen, M. & Woodley, D. T. Mechanisms of human skin cell motility. Histol Histopathol 19, 1311-24 (2004).

37. Viennet, C. et al. Contractile forces generated by striae distensae fibroblasts embedded in collagen lattices. Arch Dermatol Res 297, 10-7 (2005).

38. Duval, C., Regnier, M. & Schmidt, R. Distinct melanogenic response of human melanocytes in mono-culture, in co-culture with keratinocytes and in reconstructed epidermis, to UV exposure. Pigment Cell Res 14, 348-55 (2001).

39. Hirobe, T. How are proliferation and differentiation of melanocytes regulated? Pigment Cell Melanoma Res 24, 462-78 (2011).

40. Yamaguchi, Y. et al. Mesenchymal-epithelial interactions in the skin: increased expression of dickkopfl by palmoplantar fibroblasts inhibits melanocyte growth and differentiation. J Cell Biol 165, 275-85 (2004).

41. Yamaguchi, Y. et al. Dickkopf 1 (DKK1) regulates skin pigmentation and thickness by affecting Wnt/beta-catenin signaling in keratinocytes. FASEB J 22, 1009-20 (2008).

42. Choi, W., Kolbe, L. & Hearing, V. J. Characterization of the bioactive motif of neuregulin-1, a fibroblast-derived paracrine factor that regulates the constitutive color and the function of melanocytes in human skin. Pigment Cell Melanoma Res 25, 477-81 (2012).

43. Shishido, E., Kadono, S., Manaka, I., Kawashima, M. & Imokawa, G. The mechanism of epidermal hyperpigmentation in dermatofibroma is associated with stem cell factor and hepatocyte growth factor expression. J Invest Dermatol 117, 627-33 (2001).

44. Kihira, C., Mizutani, H., Asahi, K., Hamanaka, H. & Shimizu, M. Increased cutaneous immunoreactive stem cell factor expression and serum stem cell factor level in systemic scleroderma. J Dermatol Sci 20, 72-8 (1998).

45. Okazaki, M. et al. The mechanism of epidermal hyperpigmentation in cafe-au-lait macules of neurofibromatosis type 1 (von Recklinghausen's disease) may be associated with dermal fibroblast-derived stem cell factor and hepatocyte growth factor. Br J Dermatol 148, 689-97 (2003).

46. Cardinali, G. et al. A kindred with familial progressive hyperpigmentation-like disorder: implication of fibroblast-derived growth factors in pigmentation. Eur J Dermatol 19, 469-73 (2009).

47. Shull, M. M. et al. Targeted disruption of the mouse transforming growth factor-beta 1 gene results in multifocal inflammatory disease. Nature 359, 693-9 (1992).

48. O'Reilly, S. Role of interleukin-13 in fibrosis, particularly systemic sclerosis. Biofactors 39, 593-6 (2013).

49. Siegel, P. M. & Massague, J. Cytostatic and apoptotic actions of TGF-beta in homeostasis and cancer. Nat Rev Cancer 3, 807-21 (2003).

### Example 2

Differential Morphological and Functional Features Of Fibroblasts Explanted from Solar Lentigo.

Materials and methods

Materials

Phosphate buffer solution (PBS), Dulbecco's modified Eagle's medium (DMEM), fetal calf serum (FCS), gentamycin, trypsin and all cell culture plastics were purchased from Dutscher (Brumath, France); paraformaldehyde (PFA), 3-(4,5-dimethylthiazol-2-yl)-2,5-diphenyltetrazolium bromide (MTT), sodium dodecyl sulfate (SDS), dimethylformamide (DMF), glycine, acetone, triton X-100, bovine serum albumin (BSA), mouse anti-α-SM actin (clone 1A4, Sigma-Aldrich) and goat anti-mouse IgG-FITC antibodies, rhodamine-conjugated phalloidin, Hoechst were purchased from Sigma-Aldrich (St Quentin Fallavier, France).

Preparation and Cell Culture of Primary Fibroblasts from Skin Biopsies

Skin specimens were obtained from 10 volunteers (Caucasian females, 67-89 years of age) after written informed consent (Pr. P. Humbert, University hospital of Besançon, France). Two biopsies of 3-mm punches (20 mm apart) were collected on the back of the hand from the same patient; one containing a solar lentigo lesional macula and one excluding any visible pigmented spot (A FL and a FNL explant derived from one patient are defined in the text as couple). After surgery, both skin pieces were kept at 4° C. in a sterilized compress previously soaked in 0.9% sterile solution of sodium chloride in water. Within one hour from the sampling, the skin pieces were washed for 10 minutes with 1% penicillin/streptomycin antibiotics cocktails. Thereafter, explants were maintained in cell culture dishes containing DMEM supplemented with 10% FCS and 1% Gentamycin for fibroblasts extraction. Installed vertically to dry for 1 hour in a humidified 5% CO2 atmosphere at 37° C., cell culture dishes were then slowly horizontally settled to enable the explants to be in contact with the medium. Fibroblasts obtained from SL spot (FL) and non-pigmented skin (FNL) of 10 patients started to migrate out from the biopsies after 1 or 2 weeks. FL and FNL were maintained until the monolayer cultures reached 80% confluency and were passaged twice using a trypsin (0.05%)/EDTA (0.02%) solution. Cells were used for experiments at passages 2-7.

Fluorescence Staining

Primary FNL and FL fibroblasts were seeded in Lab-Tek chamber slides at 1×10⁴ cells/chamber and maintained in culture at 37° C. for 24 h. They were then fixed 10 min in 3% PFA solution and permeabilized for 10 min with cold acetone at −20° C. Nonspecific adhesion sites were blocked by 10 min incubation in 1% glycine solution followed by 1 h incubation in BSA solution (3% BSA/10% serum/0.1% triton X-100 in PBS). Primary antibody directed against human α-SMA was then added (1: 100 in PBS/1%BSA/0.1% triton X-100) and incubated overnight at 4° C. in a moist chamber. Cells were then incubated with the FITC conjugated secondary antibody (1: 40 in PBS) for 1 h and further 30 min with rhodamine-conjugated phalloidin for filamentous actin (F-actin) staining (2.5 μg/ml). Cellular nuclei were countermarked with Hoechst solution. Cells were then mounted in Dako fluorescent medium, observed under fluorescence microscopy (Axioskop 40, Zeiss) and further analyzed using MetaMorph software (Molecular Devices). Elliptical Fournier analysis (EFA Harmonic 2, available within the MetaMorph package) was used to describe and quantify the cellular body shape of FNL and FL. Elliptical Form Factor was calculated by the ratio of the object breadth (the caliper width of the object, perpendicular to the longest chord) and its length (the span of the longest chord through the object) for 109 FNL and 102 FL cells explanted from 5 skin couples.

Cell Proliferation

Proliferation of FNL and FL was determined by measuring metabolic activity (colorimetric MTT assay) (1) and by counting viable cells (Trypan blue exclusion assay).

MTT Assay

FNL and FL were seeded into 96-well plates (5×10³ cells/well) and incubated with DMEM supplemented for 24 h. After removing supernatant and washing by PBS 1×, 10 μl of MTT solution (0.5 mg/mL in DMEM) were added for 4 h at 37° C. The formazan-blue crystals were dissolved overnight at 37° C. in 100 μL of extraction medium (10% SDS/0.04% DMF in H₂O). The optical density was read at 570 nm with a Multiskan FC™ spectrophotometer (Fisher Scientific).

Trypan Blue Exclusion Assay

FNL and FL were seeded at a density of 7×105 cells/T75 flask and were incubated with DMEM supplemented for 4 days. Cells were trypsinized and counted by Trypan blue stain.

Senescence-Associated β-Galactosidase Activity

FNL and FL from 2 SL patients, as well as normal fibroblasts explanted from one biopsy collected after abdominal plastic surgery, were seeded at a density of 7000 cells/cm² in fibroblasts medium for 72 hours. Normal fibroblasts were subjected (FUV) or not (FNUV) to repeated-UVB exposures (200 mJ/cm²) with a VL-6.M tube (6W, 312 nm, Fischer Scientific, Massachusetts, USA). Senenescence-associated β-Galactosidase activity was detected using the SA-β-Gal staining kit (Sigma Aldrich, France) according to the manufacturer's guidelines. Fibroblasts were then analysed by phase contrast on an Olympus microscope. Fibroblasts with blue cytoplasmic staining were scored as positive. The ratio of SA-β-Gal positive cells over total cell numbers was determined by blind counting 3 fields/well in the triplicated FNL (27/514 cells), FL (87/388 cells), FNUV (5/458 cells), FUV (269/353 cells).

ELISA and V-Plex Assays

Secreted factors in paired FNL/FL-conditioned media were quantified by ELISA kits (Quantikine® for KGF, HGF, SCF and TGF-β1; R&D Systems) or by MSD 30-PLEX™ Proinflammatory Panell, Cytokine Panell, Chemokine Panell kits and TGFβ1 kit, according to the manufacturer's protocols.

Scratch Assay

Primary FNL and FL were grown on 12-well Falcon® plates for 24 h to reach 95-100% cell confluency. Scratches were done using a sterile 10 μl-pipette tip across each well. After gentle washes to remove loose cells and debris, cells were incubated with supplemented DMEM without Red phenol, covered with mineral oil and placed in the incubator at 37° C. with 5% CO2 of BD Pathway 855 Bioimaging (Becton Dickinson, USA). Image captures every hour using the AttoVision Software (Becton Dickinson) were taken for a 16 h-period. The width of the scratch was measured at 3 different locations for each time point by using ImageJ Software. The width at each time point (tn) was subtracted from the width at time (t0) and normalized to the width at time 0 ((t0−tn)/t0) and the values were expressed as percentage of scratch closure that reflects the FL and FNL migration capacity.

Statistical Analysis

Statistical significance was assessed using paired Student's t test (Prism5.0, Graph Pad Software). P values were considered significant with the following degrees: * p<0.05; ** p<0.01; *** p<0.001; **** p<0.0001.

### REFERENCES

1. Mosmann, T. Rapid colorimetric assay for cellular growth and survival: application to proliferation and cytotoxicity assays. J Immunol Methods 65, 55-63 (1983).

2. Coppe, J. P., Desprez, P. Y., Krtolica, A. & Campisi, J. The senescence-associated secretory phenotype: the dark side of tumor suppression. Annu Rev Pathol 5, 99-118 (2010).

3. Acosta, J. C. et al. A complex secretory program orchestrated by the inflammasome controls paracrine senescence. Nat Cell Bio115, 978-90 (2013).

4. Waldera Lupa, D. M. et al. Characterization of Skin Aging-Associated Secreted Proteins (SAASP) Produced by Dermal Fibroblasts Isolated from Intrinsically Aged Human Skin. J Invest Dermatol 135, 1954-68 (2015).

5. Duval, C. et al. Key regulatory role of dermal fibroblasts in pigmentation as demonstrated using a reconstructed skin model: impact of photo-aging. PLoS One 9, e114182 (2014).

Results

Upon aging, chronic exposure to ultraviolet radiations and pollution induces benign hyper-pigmented lesions, such as Solar Lentigo maculae (SL) (1). Well-defined histologically, SL is distinguishable from other hyper-pigmented diseases and can be classified relative to its evolution (2-4). Differential gene-profiling analyses comparing SL and normal skin biopsies revealed that SL tissues are mainly composed of epidermal activated melanocytes as well as hypo-proliferating and hypo-differentiated keratinocytes with a background of chronic inflammation. In absence of fibroblast markers, immuno-staining analyses for several growth factors and secreted proteins in the upper dermis of SL biopsies strongly suggest that dermal fibroblasts contribute functionally to dysregulation of epidermal cells (5). These observations are strengthened by recent studies using a pigmented reconstructed skin model that demonstrates the influence of dermal fibroblasts on skin pigmentation (6). However, data on the morphological and functional features of the SL primary fibroblasts that could explain their role in SL disease are not available.

In this study, we developed for the first time an in vitro model of SL primary fibroblasts obtained from 10 volunteers. Two biopsies, from a Solar Lentigo macula and from a non-pigmented skin respectively, were collected from the same patient. Fibroblastic explants from both biopsies (FL and its normal counterpart FNL; called FL/FLN couple) were cultured for two rounds before analysing and comparing their intra-individual morphological and functional features.

Global microscopic examination revealed that both primary fibroblasts with similar culture condition exhibit variable shape with the tendency of a more elongated appearance for the FL fibroblasts in all of the examined FL/FNL couples (data not shown). The Comparison of phalloidin stained FNL-versus FL showed that FL fibroblasts present a more intense labelling of F-actin (data not shown). This observation suggests that the two types of primary fibroblasts have different actin-based cytoskeletal frameworks. However, immunofluorescence analysis of α-SMA revealed similar amounts of this myo-fibroblastic marker in FL and FNL (FIG. 4), in agreement with a possible reticular origin 7. Our data confirm the absence of difference between peri-lesional and SL skins in terms of α-SMA-positive cells in the dermis 4 and proposes that the SL-fibroblast model shares functional features with the fibroblasts described in SL biopsies.

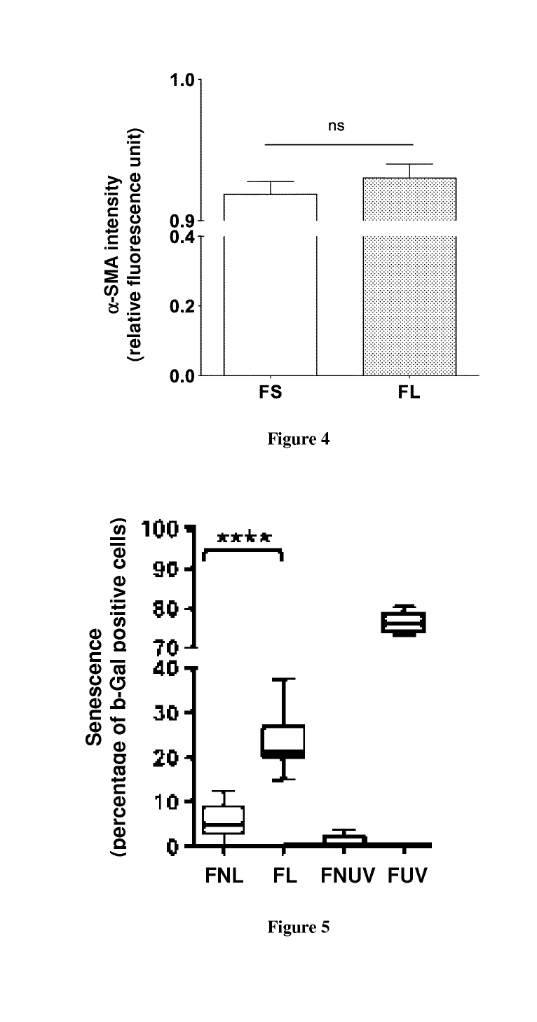

To explore functionally both types of fibroblasts, we first analysed their proliferation rate. As shown in the FIG. 2a, cell number was significantly lower in FL relative to FNL after 96 hours of culture. Moreover, FL cells showed lower metabolic activity as compared to FNL cells (FIG. 2b). Since an arrest of cell proliferation with persistency of metabolic activity is a hallmark of senescence (8), we quantified the senescence-associated β-galactosidase activity in FNL versus FL cells. As shown in FIG. 5, the percentage of β-galactosidase positive cells was significantly higher in FL compared to FNL but lower than UV-treated primary fibroblasts used as control. Because senescent cells develop altered activities that may induce changes in the tissue microenvironment (8), we characterized the secretion capacity of both types of fibroblasts by quantifying cytokines and growth factors in their respective conditioned media (CM) by ELISA and V-plex assays. As shown in FIG. 3, HGF, KGF and SCF were detected in both FNL and FL CM with significant higher levels in FL CM. These results support data revealing higher levels of these 3 factors in the dermis of the SL skin (9). The analysis of 31 additional secreted factors involved in inflammation revealed that IL-13 cytokine and growth factor TGFβ1 as significantly increased in the FL media as compared to FNL; the concentration of 29 other soluble factors was either not significantly different or undetectable. Comparison of the FL secretory profile to secretomes of senescent cells (8), fibroblasts isolated from aged skin (10) and photo-aged vs unexposed fibroblasts in an in vitro skin model (6) confirmed the senescent-like phenotype of the FL (Table 1).

Since wound healing is compromised in aging adults, we examined the migration capacity of both fibroblastic cell types by a scratch assay and calculated the percentage of the scratch closure at each time point. The results presented in FIG. 2c show that migration kinetics of FL were significantly lower than those of FNL. The FL different actin-based cytoskeletal framework (data not shown) and/or its specific pattern of secreted proteins (FIG. 3) may explain the lower FL mobility.

In conclusion, isolation of fibroblasts from adjacent- and SL-skin biopsies allowed establishing primary dermal cell models with some specific morphological and functional features that are similar to those described in SL biopsies. New investigations are now required to clarify the autocrine/paracrine roles of the differential concentrations of soluble factors on melanocyte and keratinocyte functions. It is tempting to speculate that our primary fibroblast model constitutes a valid cellular tool to test compounds for the development of whitening and anti-aging agents in topical treatments.

TABLE 1
Comparison of soluble secreted factors from fibroblasts isolated from Solar lentigo (FL) and non-lesional (FNL)
skins (this study) with those from senescent cells (SASP) 2, 3, fibroblasts isolated from intrinsically aged skin
(SAASP) 4 and from photo-aged vs young fibroblasts in an in vitro skin model5. FNL and FL-conditioned media
(n = 10 couples) were subjected to quantification of 34 soluble factors using ELISA or V-PLEX assays (Figures,
Table 1 and Materiel and Methods). Concentration of each factor was calculated by normalization to cell number.
Means of concentration (column “Mean”), standard deviations (column “ET”) and p values were calculated
using paired Student's t-test. Grey lines indicate quantified factors below detection (BD). “Δ” means variation of
the measured soluble factors (↑ = increased, [symbol] = decreased, x = no change and nd = not determined)
between FNL and FL. Comparison of the variations (Δ) of SL fibroblast associated secretory profile, SASP,
SAASP and photo-aged fibroblasts reveals that the FL secretory profile behaves as SASP.

| | | | | | | | | | Secreted |
|---|---|---|---|---|---|---|---|---|---|
| | | | | | | | | Skin | soluble factors |
| | | | | | | | | Aging- | from culture |
| | Solar Lentigo Fibroblast-associated secretory | | | | | | Senescence- | Associated | supernatants of |
| | profile (this study) | | | | | | associated | Secreted | fibroblasts from |
| | FNL | | FL | | | | phenotype | proteins | photo-aged vs young |
| Soluble | Mean | | Mean | | | | (SASP) | (SAASP) | unexposed skin |
| factors | (pg/ml) | ET | (pg/ml) | ET | p | Δ | Δ | Δ | Δ |
| HGF | 7.26E−03 | 1.37E−03 | 3.73E−02 | 5.30E−03 | 0.0003*** | ↑ | ↑ | nd | X |
| IL-13 | 4.35E−05 | 7.55E−06 | 7.60E−05 | 1.70E−05 | 0.0294* | ↑ | ↑ | X | nd |
| KGF | 5.56E−04 | 1.66E−04 | 9.15E−04 | 2.49E−04 | 0.0388* | ↑ | ↑ | X | X |
| SCF | 1.28E−03 | 3.39E−04 | 2.20E−03 | 4.41E−04 | 0.0168* | ↑ | ↑ | nd | X |
| TGFβ1 | 2.31E−03 | 2.39E−04 | 3.70E−03 | 5.17E−04 | 0.0076** | ↑ | ↑ | nd | nd |
| Eotaxin | 6.30E−05 | 5.58E−05 | 8.98E−05 | 6.69E−05 | 0.152 | X | X | X | nd |
| | | | | | ns | | | | |
| Eotaxin-3 | 8.88E−05 | 4.70E−05 | 1.38E−04 | 1.16E−04 | 0.192 | X | ↑ | nd | nd |
| | | | | | ns | | | | |
| GM- | 4.90E−06 | 3.93E−06 | 1.14E−05 | 1.42E−05 | 0.167 | X | ↑ | nd | [symbol] |
| CSF | | | | | ns | | | | |
| IFN-γ | 6.69E−06 | 4.86E−06 | 1.25E−05 | 1.15E−05 | 0.102 | X | X | ↑ | nd |
| | | | | | ns | | | | |
| IL-10 | 1.49E−06 | 9.92E−07 | 3.41E−06 | 3.93E−06 | 0.102 | X | nd | nd | nd |
| | | | | | ns | | | | |
| IL-12 | BD | | BD | | | | nd | nd | nd |
| IL- | 3.99E−06 | 2.12E−06 | 6.83E−06 | 5.93E−06 | 0.089 | X | nd | nd | nd |
| 12p70 | | | | | ns | | | | |
| IL-15 | BD | | BD | | | | ↑ | ↑ | nd |
| IL-16 | 2.45E−05 | 1.27E−05 | 3.60E−05 | 2.20E−05 | 0.116 | X | nd | nd | nd |
| | | | | | ns | | | | |
| IL-17A | 5.17E−06 | 2.21E−06 | 7.43E−06 | 3.67E−06 | 0.110 | X | nd | nd | nd |
| | | | | | ns | | | | |
| IL-1α | BD | | BD | | | | ↑ | nd | X |
| IL-1β | BD | | BD | | | | ↑ | ↑ | nd |
| IL-2 | BD | | BD | | | | nd | nd | nd |
| IL-4 | 1.13E−06 | 6.95E−07 | 1.80E−06 | 1.62E−06 | 0.133 | X | nd | ↑ | nd |
| | | | | | ns | | | | |
| IL-5 | ND | | ND | | | | nd | nd | nd |
| IL-6 | 7.54E−04 | 5.29E−04 | 1.06E−03 | 1.20E−03 | 0.258 | X | ↑ | X | X |
| | | | | | ns | | | | |
| IL-7 | 2.79E−05 | 2.24E−05 | 5.45E−05 | 5.15E−05 | 0.135 | X | ↑ | X | nd |
| | | | | | ns | | | | |
| IL-8 | 1.60E−03 | 1.67E−03 | 3.46E−03 | 4.49E−03 | 0.168 | X | ↑ | ↑ | nd |
| | | | | | ns | | | | |
| IL-8 cck | BD | | BD | | | | nd | nd | nd |
| IP-10 | 1.20E−05 | 1.02E−05 | 2.12E−05 | 2.42E−05 | 0.094 | X | nd | ↑ | nd |
| | | | | | ns | | | | |
| MCP-1 | | BD | BD | | | | ↑ | nd | X |
| MCP-4 | 2.84E−05 | 2.48E−05 | 4.54E−05 | 7.11E−05 | 0.430 | X | ↑ | nd | nd |
| | | | | | ns | | | | |
| MDC | 1.73E−04 | 1.36E−04 | 2.01E−04 | 1.12E−04 | 0.470 | X | nd | nd | nd |
| | | | | | ns | | | | |
| MIP-1α | 1.73E−04 | 1.36E−04 | 2.01E−04 | 1.12E−04 | 0.470 | X | ↑ | X | nd |
| | | | | | ns | | | | |
| MIP-1β | 1.60E−05 | 1.03E−05 | 3.13E−05 | 4.02E−05 | 0.260 | X | nd | nd | nd |
| | | | | | ns | | | | |
| TARC | 5.04E−05 | 4.53E−05 | 7.72E−05 | 4.78E−05 | 0.201 | X | nd | nd | nd |
| | | | | | ns | | | | |
| TNF-α | 2.99E−06 | 2.48E−06 | 6.17E−06 | 5.84E−06 | 0.089 | X | nd | ↑ | nd |
| | | | | | ns | | | | |
| TNF-β | BD | | BD | | | | nd | nd | nd |
| VEGF-A | 1.76E−03 | 1.09E−03 | 2.20E−03 | 1.23E−03 | 0.366 | X | ↑ | X | nd |
| | | | | | ns | | | | |

### REFERENCES

1. Nakamura, M. et al. Environment-induced lentigines: formation of solar lentigines beyond ultraviolet radiation. Exp Dermatol 24, 407-11 (2015).

2. Lin, C. B. et al. Immuno-histochemical evaluation of solar lentigines: The association of KGF/KGFR and other factors with lesion development. J Dermatol Sci 59, 91-7 (2010).

3. Goorochurn, R. et al. Biological processes in solar lentigo: insights brought by experimental models. Exp Dermatol (2016).

4. Cario-Andre, M. et al. Perilesional vs. lesional skin changes in senile lentigo. J Cutan Pathol 31, 441-7 (2004).

5. Bastonini, E., Kovacs, D. & Picardo, M. Skin Pigmentation and Pigmentary Disorders: Focus on Epidermal/Dermal Cross-Talk. Ann Dermatol 28, 279-89 (2016).

6. Duval, C. et al. Key regulatory role of dermal fibroblasts in pigmentation as demonstrated using a reconstructed skin model: impact of photo-aging. PLoS One 9, e114182 (2014).

7. Mine, S., Fortunel, N. O., Pageon, H. & Asselineau, D. Aging alters functionally human dermal papillary fibroblasts but not reticular fibroblasts: a new view of skin morphogenesis and aging. PLoS One 3, e4066 (2008).

8. Coppe, J. P., Desprez, P. Y., Krtolica, A. & Campisi, J. The senescence-associated secretory phenotype: the dark side of tumor suppression. Annu Rev Pathol 5, 99-118 (2010).

9. Kovacs, D. et al. Role of fibroblast-derived growth factors in regulating hyperpigmentation of solar lentigo. Br J Dermatol 163, 1020-7 (2010).

10. Waldera Lupa, D. M. et al. Characterization of Skin Aging-Associated Secreted Proteins (SAASP) Produced by Dermal Fibroblasts Isolated from Intrinsically Aged Human Skin. J Invest Dermatol 135, 1954-68 (2015).

1. A method of treating solar lentigo in a subject in need thereof, comprising
administering to the subject a therapeutically effective amount of an IL-13 inhibitor.

2. The method according to claim 1 wherein said IL-13 inhibitor is an IL-13 signaling pathway inhibitor.

3. The method according to claim 2 wherein said IL-13 signaling pathway inhibitor is selected from the group consisting of IL-13Rα1 antagonist, IL-13Rα1 expression inhibitor, IL-4Rα antagonist, IL-4Rα expression inhibitor, IL-13Rα2 antagonist, IL-13Rα2 expression inhibitor, JAK1 inhibitor, JAK1 expression inhibitor, JAK2 inhibitor, JAK2 expression inhibitor, STAT6 inhibitor, STAT6 expression inhibitor, TYK2 inhibitor, TYK2 expression inhibitor, TGFβ inhibitor and TGFβ expression inhibitor.

4. The method according to claim 1, wherein the IL-13 inhibitor is administered in combination with one or more of a Hepatocyte Growth Factor (HGF) inhibitor, a Keratinocyte Growth Factor (KGF) inhibitor and a Stem Cell Factor (SCF) inhibitor.

5-8. (canceled)

9. A method of screening a candidate compound for use as a drug for the treatment of solar lentigo, wherein the method comprises the steps of:
(i) providing an IL-13, one or more IL-13 receptors, and one or more IL-13 downstream effectors, or a cell, tissue sample or organism expressing the IL-13, the one or more IL-13 receptors and the one or more 1L-13 downstream effectors,
(ii) contacting the IL-13. the one or more IL-13 receptors, and the one or more IL-13 downstream effectors, or the cell, tissue sample or organism expressing the IL-13, the one or more IL-13 receptors and the one or more IL-13 downstream effectors with a candidate compound,
(iii) measuring the IL-13 activity, and
(iv) selecting positively candidate compounds that inhibit IL-13 activity.

10. A method of identifying and treating a subject having or at risk of having or developing solar lentigo, comprising the steps of
measuring in a sample obtained from said subject the expression level of IL-13;
comparing the expression level with a reference value, wherein a differential in the expression level of the IL-13 in the sample and the reference value indicates that the subject has or is at risk of having or developing solar lentigo; and
administering a therapeutically effective amount of an IL-13 inhibitor to a subject whose measurement is indicative of having or having a risk of having or developing solar lentigo.

11. The method of claim 10 further comprising a step of measuring in the sample obtained from said subject the expression level of at least one biomarker selected from the group consisting of TGFβ1, HGF, KGF and SCF.

12. (canceled)

13. The method of claim 10, further comprising monitoring the progression of solar lentigo in the subject before, during or after treatment.

14. (canceled)

15. The method of claim 9, wherein the candidate compound is a small organic molecule, a peptide, a polypeptide, an aptamer, an oligonucleotide, an antibody or an intra-antibody.

16. The method of claim 1, wherein the treatment is a cosmetic treatment of the subject.
